(19) United States
(12) Patent Application Publication
(10) Pub. No.: US 2020/0037993 A1
(43) Pub. Date: Feb. 6, 2020

(54) GENERATING A SOURCE POINT OF SHEAR WAVES FOR SHEAR WAVE ELASTICITY IMAGING

(71) Applicants: Bahador Makkiabadi, Tehran (IR); Zahra Mardi, Karaj (IR); Mahdi Beitollahi, Tehran (IR); Najmeh Meimani, Tehran (IR); Mohammad Sajad Manuchehri, Tehran (IR)

(72) Inventors: Bahador Makkiabadi, Tehran (IR); Zahra Mardi, Karaj (IR); Mahdi Beitollahi, Tehran (IR); Najmeh Meimani, Tehran (IR); Mohammad Sajad Manuchehri, Tehran (IR)

(73) Assignee: Bahador Makkiabadi, Tehran (IR)

(21) Appl. No.: 16/600,521

(22) Filed: Oct. 13, 2019

Related U.S. Application Data
(60) Provisional application No. 62/745,298, filed on Oct. 13, 2018.

(51) Int. Cl. A61B 8/08; A61B 8/00; G01S 7/52

(52) U.S. Cl. CPC A61B 8/485; A61B 8/4494; G01S 7/52085; G01S 7/52042; A61B 8/4461

(57) ABSTRACT

A method for generating a source point of shear waves for shear-wave elasticity imaging (SWEI) of an object. The method includes placing an ultrasound transducer on a surface of the object, vibrating the ultrasound transducer along a vibration axis utilizing a vibrator when the ultrasound transducer is placed on the surface of the object, and generating a shear wave in the source point by applying an acoustic radiation force on the source point. Applying the acoustic radiation force includes focusing an ultrasonic beam of the ultrasound transducer at a first focus point. The first focus point is located at the source point. The acoustic radiation force is applied simultaneously with vibrating the ultrasound transducer.

### CROSS-REFERENCE TO RELATED APPLICATION

This application claims the benefit of priority from pending U.S. Provisional Patent Application Ser. No. 62/745,298, filed on Oct. 13, 2018, and entitled “ANNULAR ARRAYS IN TRANSIENT ELASTOGRAPHY,” which is incorporated herein by reference in its entirety.

### TECHNICAL FIELD

The present disclosure generally relates to elastography, and particularly, to shear-wave elasticity imaging

### BACKGROUND

Shear wave (SW) elastography is used for imaging soft objects by inducing acoustic SWs into a body and scanning echoes of transmitted waves. Since velocity of SWs may depend on elasticity of propagation media (i.e., intended objects for imaging), elasticity (or stiffness) level of objects may be inferred by scanning velocity of SWs.

Acoustic radiation force (ARF) is a prominent way of generating acoustic SWs. ARF may create longitudinal waves using focused ultrasound beams inside object media. SWs may be generated as a result of ARF and may cause displacements inside an object under study. Since larger displacements may facilitate obtaining better quality images, it may be desired for ARF to generate more powerful waves. However, focusing high power ultrasound beams inside objects may cause safety issues such as damaging a region under study. In addition, ARF may provide one single focal point which may cause displacements inside of a limited region of an object, resulting in a narrow field of view in obtained images. There is, therefore, a need for an acoustic SW generating method that can provide considerable displacements in a wide area inside objects of interest without causing damages to objects under study.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for generating a source point of shear waves for shear-wave elasticity imaging (SWEI) of an object. An exemplary method may include placing an ultrasound transducer on a surface of the object, vibrating the ultrasound transducer utilizing a vibrator along a vibration axis when the ultrasound transducer is placed on the surface of the object, and generating a shear wave in the source point by applying an acoustic radiation force on the source point. In an exemplary embodiment, applying the acoustic radiation force may include focusing an ultrasonic beam of the ultrasound transducer at a first focus point. In an exemplary embodiment, the first focus point may be located at the source point. In an exemplary embodiment, the acoustic radiation force may be applied simultaneously with vibrating the ultrasound transducer.

In an exemplary embodiment, focusing the ultrasonic beam may include focusing a first plurality of ultrasound waves at the first focus point. In an exemplary embodiment, each of the first plurality of ultrasound waves may be propagated from a piezoelectric element of a plurality of piezoelectric elements. In an exemplary embodiment, the plurality of piezoelectric elements may be associated with an annular array transducer (AAT).

In an exemplary embodiment, the source point may be moved from the first focus point to a second focus point by focusing a second plurality of ultrasound waves at a second focus point. In an exemplary embodiment, the source point may be moved from the first focus point to a second focus point in an axial direction. In an exemplary embodiment, the second plurality of ultrasound waves may be propagated from the plurality of piezoelectric elements. In an exemplary embodiment, each of the second plurality of ultrasound waves may be propagated from a piezoelectric element of the plurality of piezoelectric elements. In an exemplary embodiment, moving the source point from the first focus point to the second focus point may include moving the source point from the first focus point to the second focus point at a supersonic speed.

In an exemplary embodiment, focusing the ultrasonic beam may further include rotating the AAT in a lateral direction at a supersonic speed utilizing a motor. In an exemplary embodiment, rotating the AAT may further include moving the source point along a predefined path by focusing the AAT at a plurality of focal points simultaneously with rotating the AAT. In an exemplary embodiment, the plurality of focal points may be located on the predefined path. In an exemplary embodiment, moving the source point along the predefined path may include moving the source point along one of an arch-shaped path or a linear path.

Other exemplary systems, methods, features and advantages of the implementations will be, or will become, apparent to one of ordinary skill in the art upon examination of the following figures and detailed description. It is intended that all such additional systems, methods, features and advantages be included within this description and this summary, be within the scope of the implementations, and be protected by the claims herein.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A shows a flowchart of a method for generating a source point of shear waves for shear-wave elasticity imaging of an object, consistent with one or more exemplary embodiments of the present disclosure.

FIG. 1B shows a flowchart for focusing an ultrasonic beam, consistent with one or more exemplary embodiments of the present disclosure.

FIG. 2 shows a system for generating a source point of shear waves for shear-wave elasticity imaging of an object, consistent with one or more exemplary embodiments of the present disclosure.

FIG. 3A shows an ultrasound transducer generating a source point in an object, consistent with one or more exemplary embodiments of the present disclosure.

FIG. 3B shows an ultrasonic beam of an ultrasound transducer focused at a first focus point, consistent with one or more exemplary embodiments of the present disclosure.

FIG. 3C shows a schematic of an annular array transducer generating a plurality of ultrasound waves, consistent with one or more exemplary embodiments of the present disclosure.

FIG. 3D shows a schematic of moving a source point in an axial direction, consistent with one or more exemplary embodiments of the present disclosure.

FIG. 3E shows a schematic of generated Mach cones when a source of acoustic radiation force moves at a supersonic speed, consistent with one or more exemplary embodiments of the present disclosure.

FIG. 3F shows a schematic of rotation of an annular array transducer, consistent with one or more exemplary embodiments of the present disclosure.

FIG. 3G shows a schematic of moving a source point along a predefined path, consistent with one or more exemplary embodiments of the present disclosure.

FIG. 3H shows a schematic of a linear path in a lateral direction, consistent with one or more exemplary embodiments of the present disclosure.

FIG. 3I shows a schematic of a linear path at an axial direction, consistent with one or more exemplary embodiments of the present disclosure.

FIG. 3J shows a schematic of an arch-shaped path, consistent with one or more exemplary embodiments of the present disclosure.

FIG. 4 shows an example computer system in which an embodiment of the present invention, or portions thereof, may be implemented as computer-readable code, consistent with exemplary embodiments of the present disclosure.

FIG. 5 shows propagation of shear waves generated by the ARF at a single focal point, consistent with one or more exemplary embodiments of the present disclosure.

FIG. 6 shows propagation of shear waves generated by ARFs at six single focal points in an axial direction, consistent with one or more exemplary embodiments of the present disclosure.

FIG. 7 shows propagation of shear waves generated by ARFs at a high supersonic speed, consistent with one or more exemplary embodiments of the present disclosure.

FIG. 8 shows propagation of shear waves generated by ARFs at six single focal points in a lateral direction, consistent with one or more exemplary embodiments of the present disclosure.

FIG. 9 shows propagation of shear waves generated by ARFs at nine single focal points through an arch-shaped path, consistent with one or more exemplary embodiments of the present disclosure.

FIG. 10 shows propagation of shear waves generated by continuous scanning of ARFs in an axial direction, consistent with one or more exemplary embodiments of the present disclosure.

FIG. 11 shows propagation of shear waves generated by continuous scanning of ARFs in a lateral direction, consistent with one or more exemplary embodiments of the present disclosure.

FIG. 12 shows propagation of shear waves generated by continuous scanning of ARFs through an arch-shaped path, consistent with one or more exemplary embodiments of the present disclosure.

FIG. 13 shows propagation of shear waves generated by continuous scanning of ARFs in an axial direction and a simultaneous application of a mechanical force, consistent with one or more exemplary embodiments of the present disclosure.

FIG. 14 shows propagation of shear waves generated by continuous scanning of ARFs in a lateral direction and a simultaneous application of a mechanical force, consistent with one or more exemplary embodiments of the present disclosure.

FIG. 15 shows propagation of shear waves generated by continuous scanning of ARFs through an arch-shaped path and a simultaneous application of a mechanical force, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein is disclosed an exemplary method and system for acoustic shear-wave (SW) generation in an SW elastography platform. An exemplary method aims to provide considerable displacements (i.e., displacements that may be detected by a scanner for imaging) in a wide area inside an object without causing safety issues, such as damaging an imaged region. To do so, an exemplary annular array transducer (AAT) may be utilized for generation of an acoustic radiation force (ARF). An exemplary AAT may be equipped with a vibrator that may apply a mechanical force to the object via vibrating the AAT. An exemplary mechanical force may lead to generation of an SW. Simultaneous application of ARF and vibration may generate strong SWs that may lead to considerable displacements inside an object of interest. An exemplary AAT may further be equipped with a motor that may rotate the AAT. Rotation of the AAT in various directions by the motor may allow generation of SWs in a wide region inside an object of interest.

An exemplary AAT may include a number of transducers with different sizes that may enable the AAT to produce ARFs toward various focal points to generate a moving source of ARF. By moving an exemplary source of ARF at a supersonic speed (i.e., a speed more than a propagation speed of SWs), generated SWs may constructively interact with each other, and consequently, generate powerful Mach cones (i.e., conical pressure wave fronts generated by a source of ARF). Exemplary Mach cones may act as a second source of SWs, resulting in larger displacements in an object of interest without applying a high power that may be harmful to the object. By simultaneous application of the above three techniques (i.e., vibration, rotation, and focus), an exemplary source of ARF may be moved in a predefined arrangement (e.g., axial, lateral, and arch-shaped). As a result, Mach cones with various patterns may be generated, and consequently, various patterns of displacements (i.e., displacements with different intensities in various regions inside an object of interest) may be obtained. Therefore, by generating displacements with relatively higher intensities, an imaging system may provide better quality images.

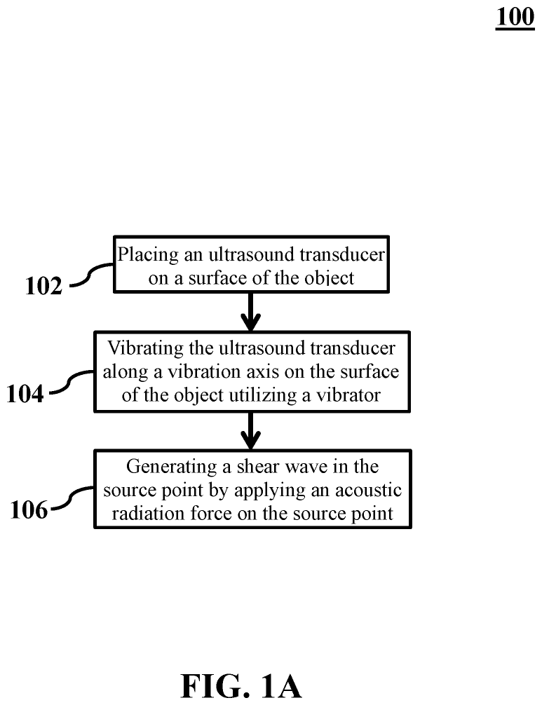

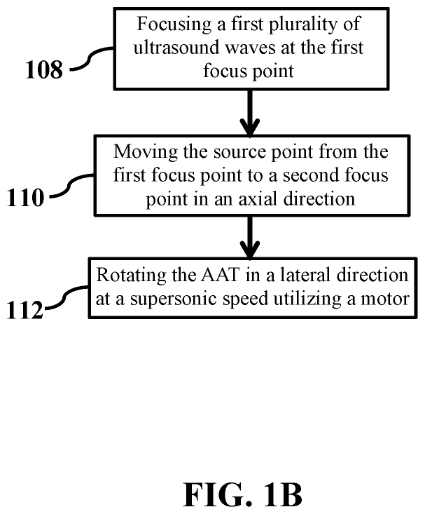

FIG. 1A shows a flowchart of a method for generating a source point of shear waves for shear-wave elasticity imaging (SWEI) of an object, consistent with one or more exemplary embodiments of the present disclosure. An exemplary method 100 may include placing an ultrasound transducer on a surface of an object (step 102), vibrating the ultrasound transducer along a vibration axis utilizing a vibrator when the ultrasound transducer is placed on the surface of the object (step 104), and generating a shear wave in a source point by applying an acoustic radiation force on the source point (step 106). An exemplary object may include a soft tissue of human body, such as liver and bile ducts. An exemplary source point may include an origin of propagation of a shear wave inside an exemplary object. In an exemplary embodiment, applying the acoustic radiation force may include focusing an ultrasonic beam of the ultrasound transducer at a first focus point. An exemplary focus point may include a desired location inside an exemplary object at which acoustic radiation force may be applied to obtain a high resolution image. Therefore, in an exemplary embodiment, the ultrasonic beam may be focused at the first focus point to obtain a higher resolution at the first focus point in an elasticity image of the object. In an exemplary embodiment, the first focus point may be located at the source point. In an exemplary embodiment, the acoustic radiation force may be applied simultaneously with vibrating the ultrasound transducer.

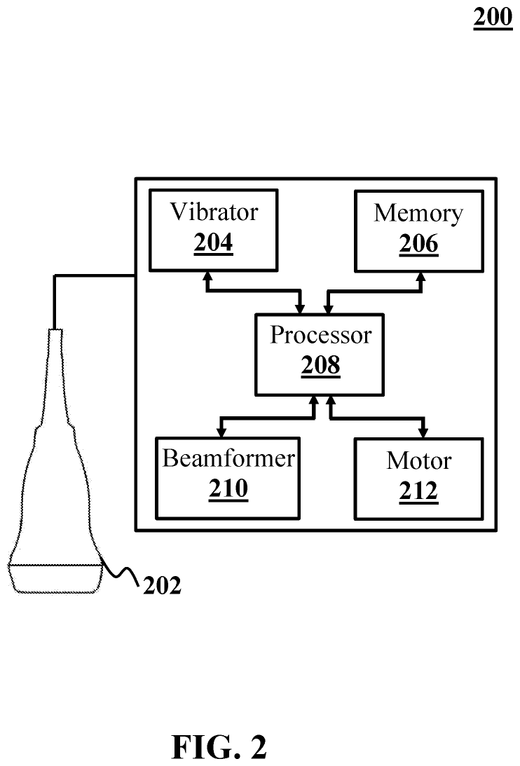

FIG. 2 shows a system for generating a source point of shear waves for SWEI of an object, consistent with one or more exemplary embodiments of the present disclosure. In an exemplary embodiment, different steps of method 100 may be implemented by utilizing an exemplary system 200. In an exemplary embodiment, system 200 may include an ultrasound transducer 202, a vibrator 204, a memory 206, and a processor 208. In an exemplary embodiment, vibrator 204 may be coupled with ultrasound transducer 202. In an exemplary embodiment, memory 206 may have processor-readable instructions stored therein. In an exemplary embodiment, processor 208 may be configured to access the memory and execute the processor-readable instructions. In an exemplary embodiment, the processor-readable instructions may include steps 104 and 106.

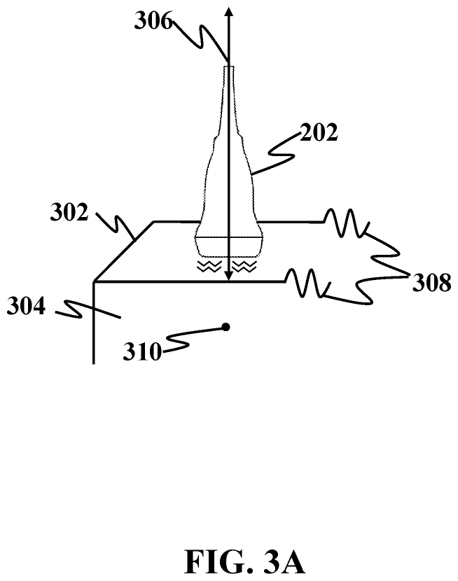

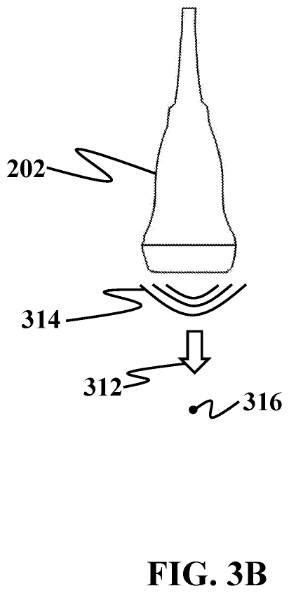

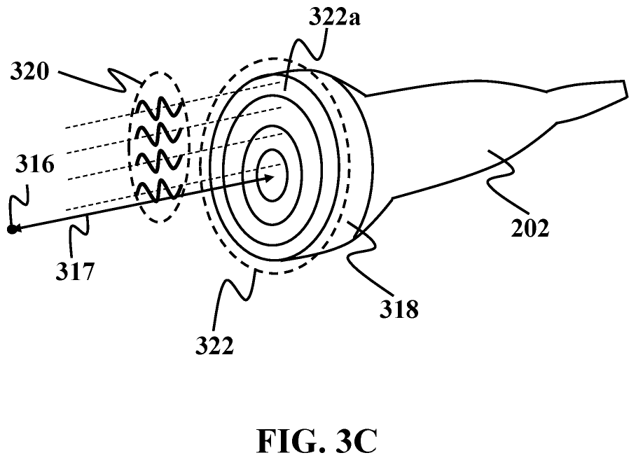

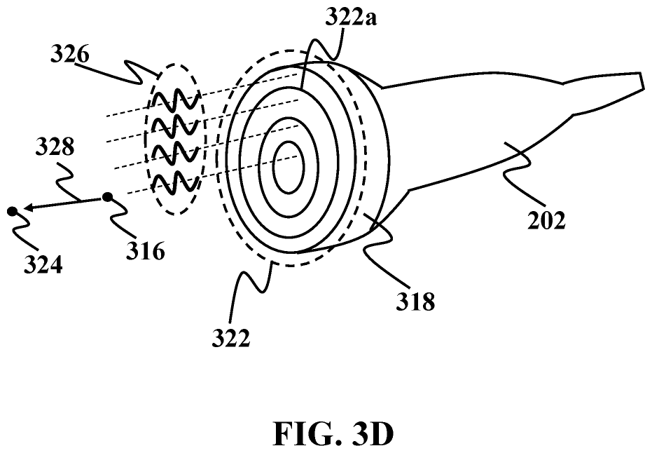

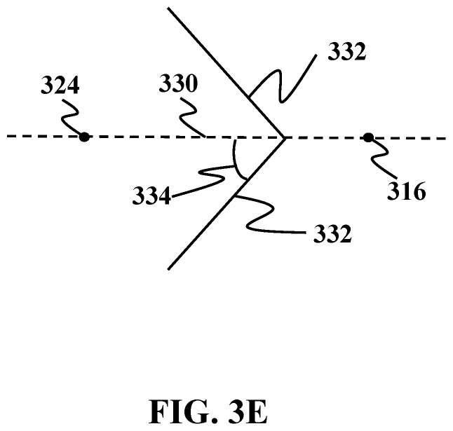

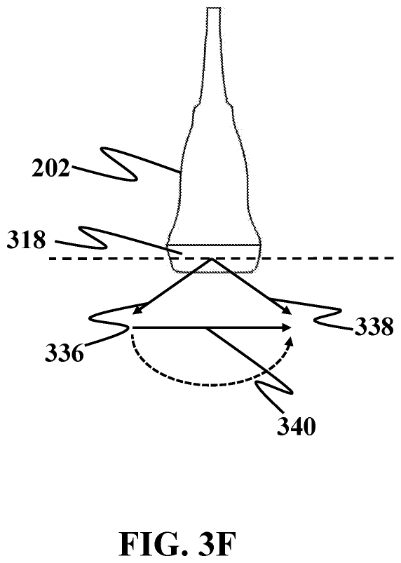

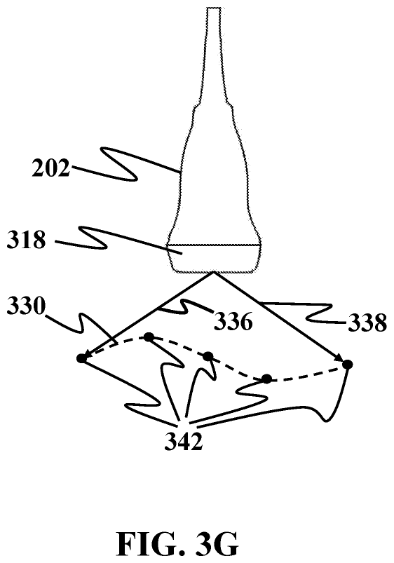

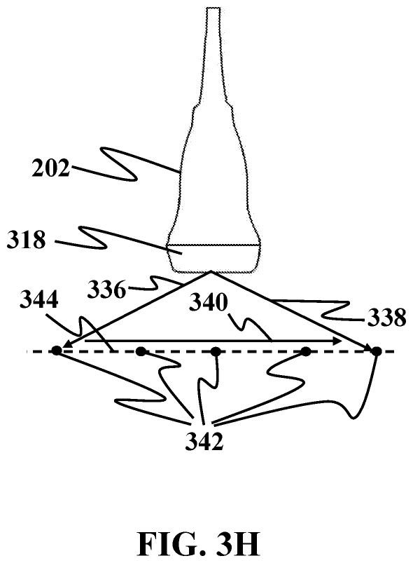

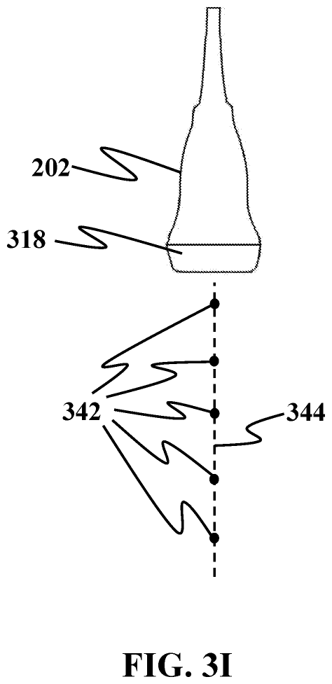

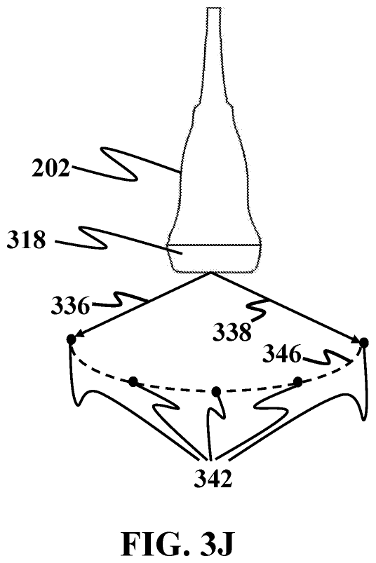

For further detail with regards to steps 102 and 104, FIG. 3A shows an ultrasound transducer generating a source point in an object, consistent with one or more exemplary embodiments of the present disclosure. FIG. 3B shows an ultrasonic beam of an ultrasound transducer focused at a first focus point, consistent with one or more exemplary embodiments of the present disclosure. Referring to FIGS. 2-3B, in an exemplary embodiment, ultrasound transducer 202 may be placed on a surface 302 of an object 304. In an exemplary embodiment, ultrasound transducer 202 may be vibrated along a vibration axis 306 utilizing vibrator 204. In an exemplary embodiment, ultrasound transducer 202 may be vibrated when ultrasound transducer 202 is placed on a surface 302 of object 304. In an exemplary embodiment, vibrator 204 may include a crankshaft. The crankshaft may be utilized to convert a circular motion to a linear motion. An exemplary linear motion may be applied to ultrasound transducer 202. In an exemplary embodiment, the linear motion may be generated in a direction parallel with vibration axis 306. In an exemplary embodiment, vibration of ultrasound transducer 202 may apply a mechanical force to object 304. In an exemplary embodiment, the mechanical force may generate a shear wave 308 inside object 304.

In an exemplary embodiment, a shear wave 308 may be generated at a source point 310 by applying an acoustic radiation force 312 at source point 310. In an exemplary embodiment, applying acoustic radiation force 312 may include focusing an ultrasonic beam 314 of ultrasound transducer 202 at a first focus point 316. In an exemplary embodiment, first focus point 316 may be located at source point 310. In an exemplary embodiment, focusing ultrasonic beam 314 at first focus point 316 simultaneous with vibration of ultrasound transducer 202 may lead to amplification of generated shear wave 308.

In further detail with respect to step 106, FIG. 1B shows a flowchart for focusing an ultrasonic beam, consistent with one or more exemplary embodiments of the present disclosure. In an exemplary embodiment, focusing the ultrasonic beam in step 106 may include focusing a first plurality of ultrasound waves at a first focus point (step 108) and moving a source point from the first focus point to a second focus point in an axial direction (step 110). In an exemplary embodiment, the source point may be moved to additional exemplary focus points inside a field of view of ultrasound transducer 202. In an exemplary embodiment, each of the first plurality of ultrasound waves may be propagated from a piezoelectric element of a plurality of piezoelectric elements. In an exemplary embodiment, the plurality of piezoelectric elements may be placed in a co-axial arrangement and may be aligned in a same plane. In an exemplary embodiment, the plurality of piezoelectric elements may form an annular array transducer (AAT). In an exemplary embodiment, the AAT may be an ultrasound transducer. In an exemplary embodiment, the source point may be moved from the first focus point to the second focus point by focusing a second plurality of ultrasound waves at the second focus point. In an exemplary embodiment, the second plurality of ultrasound waves may be transmitted from the plurality of piezoelectric elements. In an exemplary embodiment, the first focus point may be similar to first focus point 316. In an exemplary embodiment, the source point may be similar to source point 310. In an exemplary embodiment, the ultrasound transducer may be similar to ultrasound transducer 202.

In further detail with respect to step 108, FIG. 3C shows a schematic of an AAT generating a plurality of ultrasound waves, consistent with one or more exemplary embodiments of the present disclosure. Referring to FIGS. 3B and 3C, in an exemplary embodiment, an AAT 318 may generate a first plurality of ultrasound waves 320 to focus ultrasonic beam 314 at first focus point 316 with a focal distance 317. In an exemplary embodiment, focal distance 317 may be determined based on a depth of a region of interest inside object 304. In an exemplary embodiment, first plurality of ultrasound waves 320 may be generated by AAT 318. In an exemplary embodiment, AAT 318 may include a plurality of piezoelectric elements 322. In an exemplary embodiment, each of first plurality of ultrasound waves 320 may be generated by a piezoelectric element (for example, piezoelectric element 322a in FIG. 3C) of plurality of piezoelectric elements 322.

Referring to FIGS. 2, 3B, and 3C, in an exemplary embodiment, ultrasonic beam 314 may be focused at first focus point 316 by propagating first plurality of ultrasound waves 320 with a first excitation scheme. In an exemplary embodiment, the first excitation scheme may include a first predefined set of excitation delays. In an exemplary embodiment, each of first plurality of ultrasound waves 320 may be generated by exciting each piezoelectric element (for example, piezoelectric element 322a in FIG. 3C) of plurality of piezoelectric elements 322 based on the excitation delays. In an exemplary embodiment, system 200 may further include a beamformer 210 that may be configured to excite each of plurality of piezoelectric elements 322. In an exemplary embodiment, beamformer 210 may focus ultrasonic beam 314 at first focus point 316 by exciting each of plurality of piezoelectric elements 322 with a separate delay of the first predefined set of excitation delays. In an exemplary embodiment, the first excitation scheme may be stored in memory 206 and may be sent to beamformer 210 by processor 208.

In further detail with respect to step 110, FIG. 3D shows a schematic of moving a source point in an axial direction, consistent with one or more exemplary embodiments of the present disclosure. Referring to FIGS. 3A and 3D, in an exemplary embodiment, source point 310 may be moved from first focus point 316 to a second focus point 324 by focusing a second plurality of ultrasound waves 326 at second focus point 324. In an exemplary embodiment, source point 310 may be moved from first focus point 316 to a second focus point 324 in an axial direction 328. In an exemplary embodiment, second plurality of ultrasound waves 326 may be propagated from plurality of piezoelectric elements 322. In an exemplary embodiment, each of second plurality of ultrasound waves 326 may be propagated from a piezoelectric element (for example, piezoelectric element 322a) of plurality of piezoelectric elements 322. In an exemplary embodiment, axial direction 328 may be parallel to vibration axis 306.

Referring to FIGS. 2, 3B, and 3D, in an exemplary embodiment, ultrasonic beam 314 may be focused at second focus point 324 by propagating second plurality of ultrasound waves 326 with a second excitation scheme. In an exemplary embodiment, the second excitation scheme may include a second predefined set of excitation delays. In an exemplary embodiment, each of first plurality of ultrasound waves 320 may be generated by exciting each piezoelectric element (for example, piezoelectric element 322a in FIG. 3C) of plurality of piezoelectric elements 322 based on the excitation delays. In an exemplary embodiment, beamformer 210 may focus ultrasonic beam 314 at second focus point 324by exciting each of plurality of plurality of piezoelectric elements 322 with a separate delay of the second predefined set of excitation delays. In an exemplary embodiment, the second excitation scheme may be stored in memory 206 and may be sent to beamformer 210 by processor 208. In an exemplary embodiment, source point 310 may be set at axial direction 328 because of AAT 318 symmetry about axial direction 328.

In an exemplary embodiment, moving source point 310 from first focus point 316 to second focus point 324 may include moving source point 310 from first focus point 316 to second focus point 324 at a supersonic speed. In an exemplary embodiment, the supersonic speed may refer to a speed that is greater than a propagation speed of shear wave 308 through object 304. In an exemplary embodiment, when source point 310 moves at the supersonic speed, a plurality of shear waves (similar to shear wave 308) may constructively interact and generate a powerful Mach cone. Mach cones include conical pressure wave fronts that may move away and recede in time.

FIG. 3E shows a schematic of generated Mach cones when a source of acoustic radiation force moves at a supersonic speed, consistent with one or more exemplary embodiments of the present disclosure. Referring to FIGS. 3A and 3E, in an exemplary embodiment, source point 310 may move from first focus point 316 toward second focus point 324 in a predefined path 330. In an exemplary embodiment, when a moving speed of source point 310 is greater than a propagation speed of shear wave 308 through object 304, a Mach cone 332 may be generated. In an exemplary embodiment, Mach cone 332 may have a Mach angle 334 with predefined path 330. In an exemplary embodiment, Mach angle 334 may have an inverse relationship with a moving speed of source point 310. In an exemplary embodiment, Mach cone 332 may act as a second source of shear waves (similar to shear wave 308), resulting in larger displacements in object 304 without applying a high power, thereby preventing a damage to object 304. In an exemplary embodiment, moving source point 310 at a supersonic speed simultaneously with a vibration of ultrasound transducer 202 may lead to even stronger Mach cones.

Referring again to FIG. 1B, in an exemplary embodiment, focusing the ultrasonic beam in step 106 may further include rotating the AAT in a lateral direction at a supersonic speed utilizing a motor (step 112). Referring again to FIGS. 2 and 3D, in an exemplary embodiment, the AAT may be similar to AAT 318. In an exemplary embodiment, system 200 may further include a motor 212. In an exemplary embodiment, motor 212 may be configured to rotate AAT 318 in a lateral direction at a supersonic speed. In an exemplary embodiment, rotation of AAT 318 may facilitate extraction of elasticity information of object 304 from various directions. In an exemplary embodiment, extraction of elasticity information of object 304 may be performed by an ultrasound pulse echo scanner. An exemplary ultrasound pulse echo scanner may receive echoes of transmitted shear waves and may convert received echoes to electrical signals. In an exemplary embodiment, the ultrasound pulse echo scanner may include AAT 318.

In an exemplary embodiment, processor 208 may require knowing an angular position of motor 212 (which may be equivalent with an angular position of AAT 318). In an exemplary embodiment, motor 212 may include a stepper motor. In an exemplary embodiment, the stepper motor may provide processor 208 the angular position of AAT 318. In an exemplary embodiment, the stepper motor may be connected to plurality of piezoelectric elements 322 via a gear. In an exemplary embodiment, the stepper motor and the gear may be kept in a container filled with oil. In an exemplary embodiment, motor 212 may include a direct current (DC) motor. In an exemplary embodiment, the DC motor may include a shaft encoder. In an exemplary embodiment, when the DC motor is utilized, the angular position of AAT 318 may be fed back to processor 208 utilizing the shaft encoder. In an exemplary embodiment, the shaft encoder may convert the angular position of motor 212 to analog or digital outputs. In an exemplary embodiment, the output of the shaft encoder may be fed back to processor 208.

FIG. 3F shows a schematic of rotation of an AAT, consistent with one or more exemplary embodiments of the present disclosure. In an exemplary embodiment, AAT 318 may be rotated from a first direction 336 to a second direction 338. In an exemplary embodiment, AAT 318 may enable ultrasound transducer 202 to focus source point 310 at axial direction 328. In an exemplary embodiment, motor 212 may allow for moving source point 310 in a lateral direction 340. Therefore, in an exemplary embodiment, simultaneous application of focusing source point 310 and rotation of AAT 318 by motor 212 may allow for generating source point 310 at any position in object 304. In an exemplary embodiment, AAT 318 may be rotated by motor 212 at a supersonic speed, thereby generating Mach cone 332.

FIG. 3G shows a schematic of moving a source point along a predefined path, consistent with one or more exemplary embodiments of the present disclosure. In an exemplary embodiment, rotating AAT 318 may further include moving source point 310 along predefined path 330 by focusing AAT 318 at a plurality of focal points 342 simultaneously with rotating AAT 318. In an exemplary embodiment, plurality of focal points 342 may be located on predefined path 330. In an exemplary embodiment, beamformer 210 may be utilized for focusing AAT 318 at each of plurality of focal points 342. In an exemplary embodiment, AAT 318 may sweep predefined path 330 by discrete scanning of plurality of focal points 342. In other words, focal points may be spatially separated along an exemplary predefined path. In an exemplary embodiment, AAT 318 may sweep predefined path 330 by continuous scanning of plurality of focal points 342. In other words, focal points may adhere to each other along an exemplary predefined path. In an exemplary embodiment, a field of view of AAT 318 may include a region scanned by AAT 318 during a rotation of AAT 318 by motor 212Error! Bookmark not defined. Error! Bookmark not defined. from first direction 336 to second direction 338.

In an exemplary embodiment, moving source point 310 along predefined path 330 may include moving source point 310 along one of an arch-shaped path or a linear path. Referring to FIGS. 3D and 3F, in an exemplary embodiment, the linear path may be in one of linear path 344 or axial direction 328.

FIG. 3H shows a schematic of a linear path in a lateral direction, consistent with one or more exemplary embodiments of the present disclosure. Referring to FIGS. 3D-31I, in an exemplary embodiment, plurality of focal points 342 may be arranged in a linear path 344. In an exemplary embodiment, linear path 344 may be in lateral direction 340. In an exemplary embodiment, to arrange plurality of focal points 342 in linear path 344, AAT 318 may focus source point 310 in first direction 336 with a long focal distance. Then, in an exemplary embodiment, AAT 318 may be rotated by motor 212 and may simultaneously change the focal distance utilizing beamformer 210 until source point 310 reaches second direction 338, thereby maintaining plurality of focal points 342 on linear path 344. In an exemplary embodiment, arranging plurality of focal points 342 in linear path 344 in lateral direction 340 at a supersonic speed may generate Mach cone 332 in axial direction 328. In an exemplary embodiment, Mach cone 332 may generate shear wave 308 which in turn may generate displacements in object 304. In an exemplary embodiment, rotation of AAT 318 may also facilitate extraction of elasticity information of object 304 from various directions.

FIG. 3I shows a schematic of a linear path at an axial direction, consistent with one or more exemplary embodiments of the present disclosure. Referring to FIGS. 3D and 31, in an exemplary embodiment, plurality of focal points 342 may be arranged in a linear path 344. In an exemplary embodiment, linear path 344 may be in axial direction 328. In an exemplary embodiment, to arrange plurality of focal points 342 in linear path 344 in axial direction 328, AAT 318 may need to focus source point 310 at a short focal distance at first. Then, in an exemplary embodiment, AAT 318 may increase the focal distance until an end of linear path 344 is reached. In an exemplary embodiment, arranging plurality of focal points 342 in linear path 344 at axial direction 328 at the supersonic speed may generate Mach cone 332 in lateral direction 340. In an exemplary embodiment, Mach cone 332 may generate shear wave 308 which in turn may generate displacements in object 304. In an exemplary embodiment, rotation of AAT 318 may facilitate extraction of the elasticity information of object 304 from various directions.

FIG. 3J shows a schematic of an arch-shaped path, consistent with one or more exemplary embodiments of the present disclosure. In an exemplary embodiment, plurality of focal points 342 may be arranged in an arch-shaped path 346. In an exemplary embodiment, to arrange plurality of focal points 342 in an arch-shaped path 346, AAT 318 may focus at a constant focal distance. In an exemplary embodiment, to sweep from a beginning of arch-shaped path 346 to an end of arch-shaped path 346, AAT 318 may be rotated by motor 212 from first direction 336 to second direction 338. In an exemplary embodiment, arranging plurality of focal points 342 in arch-shaped path 346 at a supersonic speed may generate Mach cone 332. In an exemplary embodiment, Mach cone 332 may generate shear wave 308 which in turn generate displacements in object 304. In an exemplary embodiment, rotation of AAT 318 may also facilitate extraction of the elasticity information of object 304 from various directions.

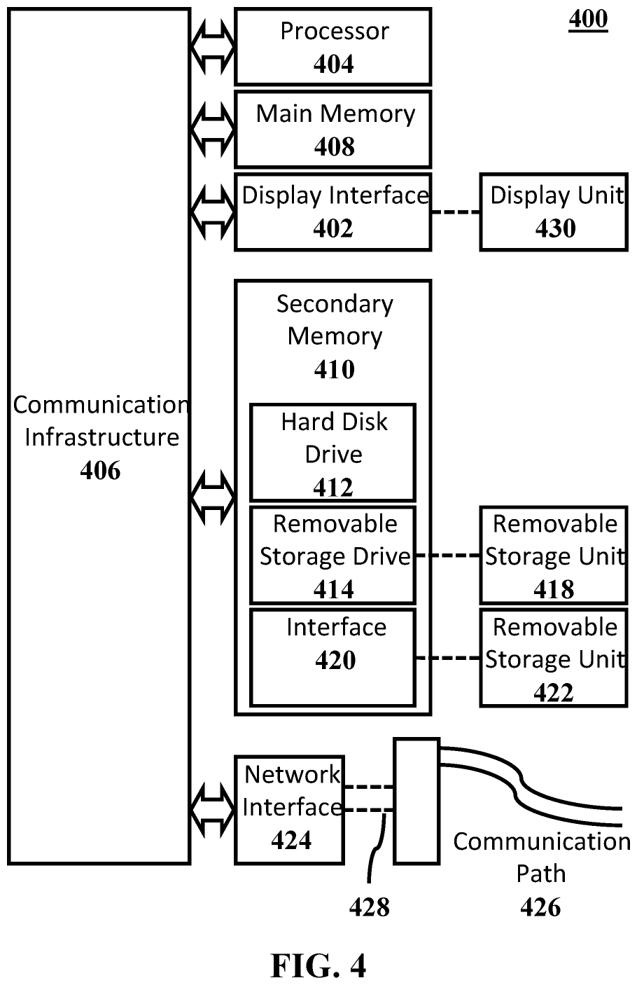

FIG. 4 shows an example computer system 400 in which an embodiment of the present invention, or portions thereof, may be implemented as computer-readable code, consistent with exemplary embodiments of the present disclosure. For example, method 100 may be implemented in computer system 400 using hardware, software, firmware, tangible computer readable media having instructions stored thereon, or a combination thereof and may be implemented in one or more computer systems or other processing systems. Hardware, software, or any combination of such may embody any of the modules and components in FIGS. 1A-3J.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this example computer system 400. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 404 may be a special purpose or a general-purpose processor device. As will be appreciated by persons skilled in the relevant art, processor device 404 may also be a single processor in a multi-core/multiprocessor system, such system operating alone, or in a cluster of computing devices operating in a cluster or server farm. Processor device 404 may be connected to a communication infrastructure 406, for example, a bus, message queue, network, or multi-core message-passing scheme.

In an exemplary embodiment, computer system 400 may include a display interface 402, for example a video connector, to transfer data to a display unit 430, for example, a monitor. Computer system 400 may also include a main memory 408, for example, random access memory (RAM), and may also include a secondary memory 410. Secondary memory 410 may include, for example, a hard disk drive 412, and a removable storage drive 414. Removable storage drive 414 may include a floppy disk drive, a magnetic tape drive, an optical disk drive, a flash memory, or the like. Removable storage drive 414 may read from and/or write to a removable storage unit 418 in a well-known manner. Removable storage unit 418 may include a floppy disk, a magnetic tape, an optical disk, etc., which may be read by and written to by removable storage drive 414. As will be appreciated by persons skilled in the relevant art, removable storage unit 418 may include a computer usable storage medium having stored therein computer software and/or data.

In alternative implementations, secondary memory 410 may include other similar means for allowing computer programs or other instructions to be loaded into computer system 400. Such means may include, for example, a removable storage unit 422 and an interface 420. Examples of such means may include a program cartridge and cartridge interface (such as that found in video game devices), a removable memory chip (such as an EPROM, or PROM) and associated socket, and other removable storage units 422 and interfaces 420 which allow software and data to be transferred from removable storage unit 422 to computer system 400.

Computer system 400 may also include a communications interface 424. Communications interface 424 allows software and data to be transferred between computer system 400 and external devices. Communications interface 424 may include a modem, a network interface (such as an Ethernet card), a communications port, a PCMCIA slot and card, or the like. Software and data transferred via communications interface 424 may be in the form of signals, which may be electronic, electromagnetic, optical, or other signals capable of being received by communications interface 424. These signals may be provided to communications interface 424 via a communications path 426. Communications path 426 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 418, removable storage unit 422, and a hard disk installed in hard disk drive 412. Computer program medium and computer usable medium may also refer to memories, such as main memory 408 and secondary memory 410, which may be memory semiconductors (e.g. DRAMs, etc.).

Computer programs (also called computer control logic) are stored in main memory 508 and/or secondary memory 410. Computer programs may also be received via communications interface 424. Such computer programs, when executed, enable computer system 400 to implement different embodiments of the present disclosure as discussed herein. In particular, the computer programs, when executed, enable processor device 404 to implement the processes of the present disclosure, such as the operations in method 100 illustrated by flowchart 100 of FIG. 1A and flowchart 112 of FIG. 1B discussed above. Accordingly, such computer programs represent controllers of computer system 400. Where an exemplary embodiment of method 100 is implemented using software, the software may be stored in a computer program product and loaded into computer system 400 using removable storage drive 414, interface 420, and hard disk drive 412, or communications interface 424.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

### EXAMPLE 1

In this example, propagation of shear waves generated by an ARF at a single focal point is illustrated. The ARF is generated by an exemplary SWEI system (analogous to system 200). The SWEI system includes an ultrasound transducer (analogous to ultrasound transducer 202), a vibrator (analogous to vibrator 204), a memory (analogous to memory 206), a processor (analogous to processor 208), a beamformer (analogous to beamformer 210), and a motor (analogous to motor 212). The ultrasound transducer includes an annular array transducer (analogous to AAT 318). The ultrasound transducer applies an acoustic radiation force (analogous to acoustic radiation force 312) to an exemplary object (analogous to object 304). The value of displacements are obtained by computer simulations. The computer simulations are based on a forward problem of displacement measurement from an acoustic wave equation. Finite difference method is utilized for solving the acoustic wave equation.

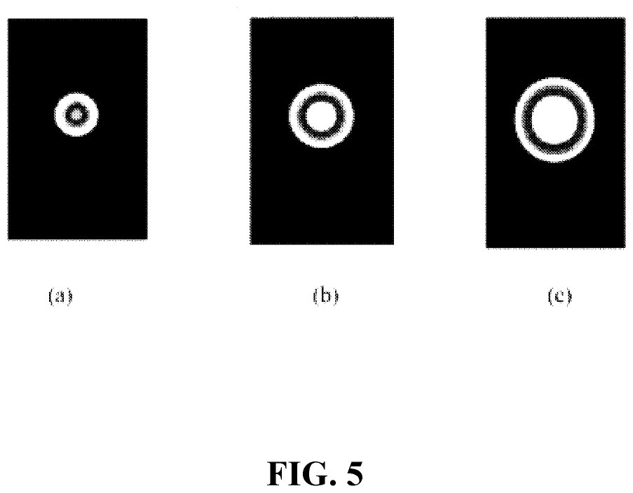

FIG. 5 shows propagation of shear waves generated by the ARF at a single focal point, consistent with one or more exemplary embodiments of the present disclosure. Subfigures (a) to (c) show propagation of generated shear waves at different moments. In this example, no vibration or rotation is applied to the ultrasound transducer. As FIG. 5 shows, the shear waves propagate in a circular pattern. Also, the shear waves cause circular displacements in a larger area as time passes. However, the intensity of propagated shear waves are low when ARF is applied at a single focal point.

### EXAMPLE 2

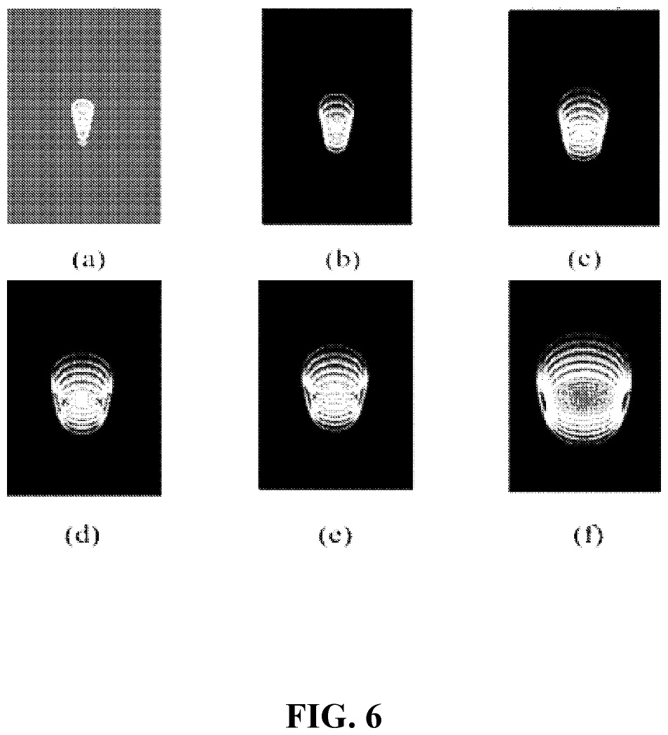

In this example, propagation of shear waves generated by ARFs at six focal points in an axial direction is illustrated. An exemplary SWEI system, similar to the SWEI system of EXAMPLE 1, is implemented in which an ultrasound transducer applies ARFs at six focal points. FIG. 6 shows propagation of shear waves generated by ARFs at six single focal points in an axial direction, consistent with one or more exemplary embodiments of the present disclosure. The focal points are arranged equally apart in an axial direction (analogous to axial direction 328) at a focal distance (analogous to focal distance 317) of about 35 mm to about 50 mm. The ultrasound transducer applies ARF at focal points at a supersonic speed. Subfigures (a) to (f) show propagation of generated shear waves and associated Mach cones at different moments. As FIG. 6 shows, Mach cones are generated in an axial direction and propagate in a lateral direction (analogous to lateral direction 340) through time. It can be observed that higher displacements are generated in comparison with the results of EXAMPLE 1. Specifically, a maximum displacement of about 12 μm is achieved for six focal points is, which degrades to about 6 μm as time passes. On the other hand, a maximum displacement of about 4 μm is obtained for a single focal point (as shown in FIG. 5) that eventually degrades to about 2 μm.

### EXAMPLE 3

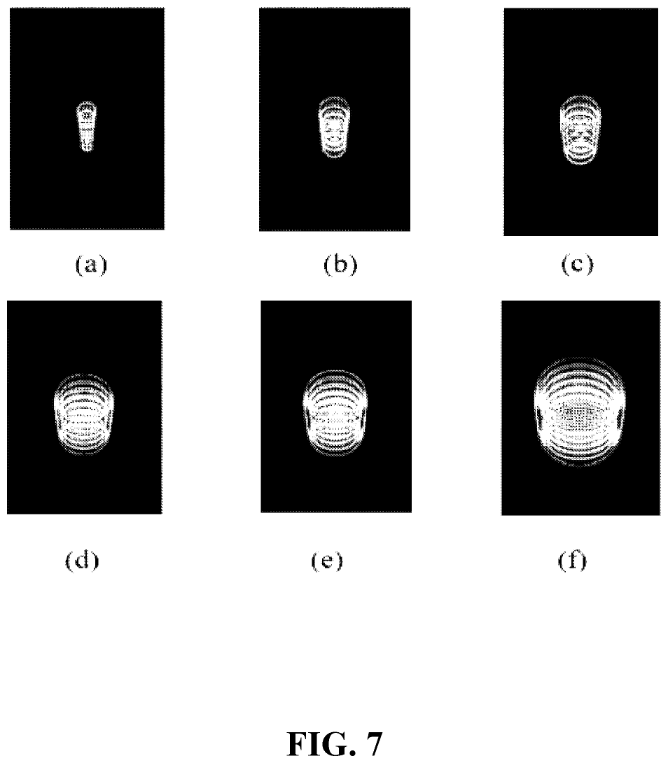

In this example, propagation of shear waves generated by ARFs at six focal points at a high supersonic speed is illustrated. An exemplary SWEI system, similar to the SWEI system of EXAMPLE 2, is implemented in which an ultrasound transducer applies ARFs at six focal points at a supersonic speed twice as that of EXAMPLE 2. FIG. 7 shows propagation of shear waves generated by ARFs at a high supersonic speed, consistent with one or more exemplary embodiments of the present disclosure. Subfigures (a) to (f) show propagation of generated shear waves and associated Mach cones at different moments. As it can be observed, a Mach angle (analogous to Mach angle 334) reduces compared to that of FIG. 5 due to an increase in speed of generation of shear waves.

### EXAMPLE 4

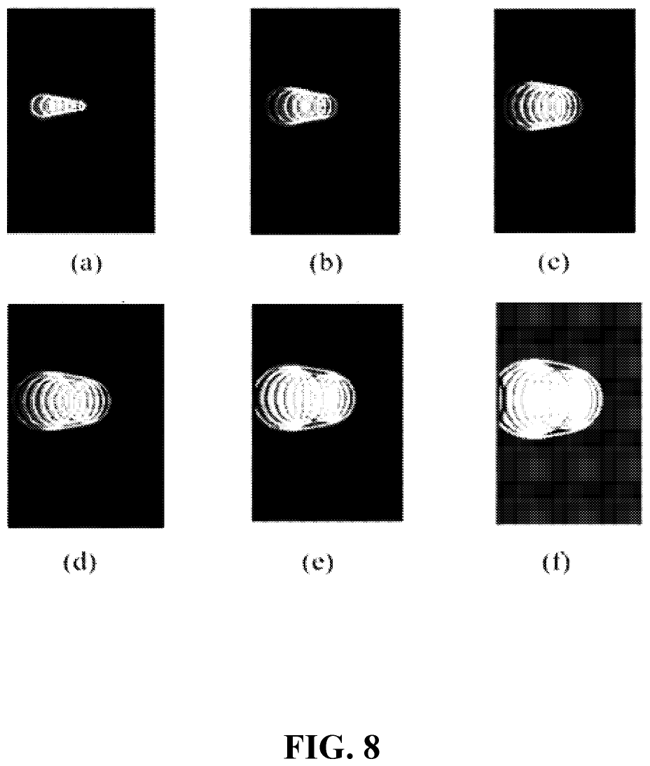

In this example, propagation of shear waves generated by ARFs at six focal points at a lateral direction is illustrated. An exemplary SWEI system, similar to the SWEI system of EXAMPLE 1, is implemented in which an ultrasound transducer applies ARFs at six focal points. FIG. 8 shows propagation of shear waves generated by ARFs at six single focal points in a lateral direction, consistent with one or more exemplary embodiments of the present disclosure. The focal points are arranged equally apart with about 2.5 mm distance in a lateral direction (analogous to lateral direction 340). The ultrasound transducer applies ARFs at focal points at a supersonic speed with the aid of a motor (analogous to motor 212). Subfigures (a) to (f) show propagation of generated shear waves and associated Mach cones at different moments. As FIG. 8 shows, Mach cones are generated in a lateral direction and propagate in an axial direction (analogous to axial direction 328) through time.

### EXAMPLE 5

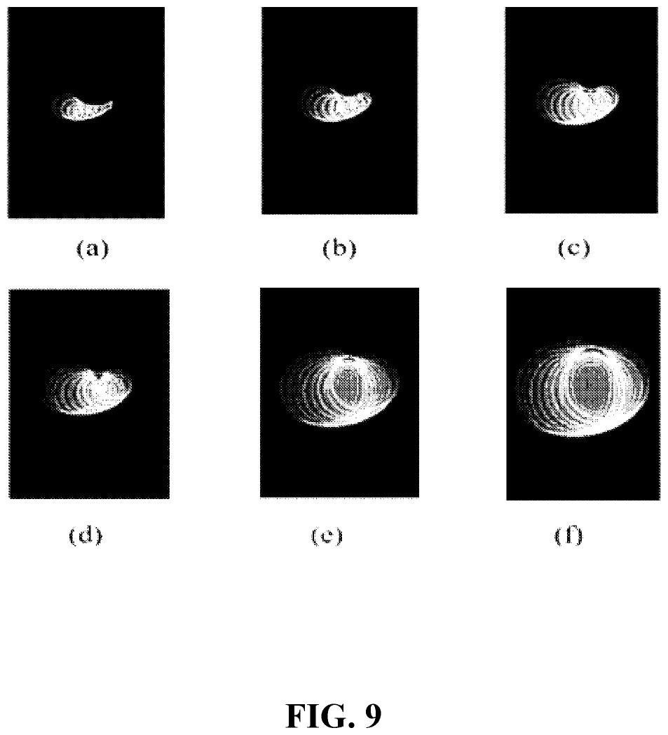

In this example, propagation of shear waves generated by ARFs at nine focal points through an arch-shaped path is illustrated. An exemplary SWEI system, similar to the SWEI system of EXAMPLE 1, is implemented in which an ultrasound transducer applies ARFs at nine focal points. FIG. 9 shows propagation of shear waves generated by ARFs at nine single focal points through an arch-shaped path, consistent with one or more exemplary embodiments of the present disclosure. Subfigures (a) to (f) show propagation of generated shear waves and associated Mach cones at different moments. The ultrasound transducer applies nine ARFs with a fixed focal distance and an arch-shaped path (analogous to arch-shaped path 346) is created by rotating the ultrasound transducer. As FIG. 9 shows, generated Mach cones propagate in a special pattern similar to a crescent.

### EXAMPLE 6

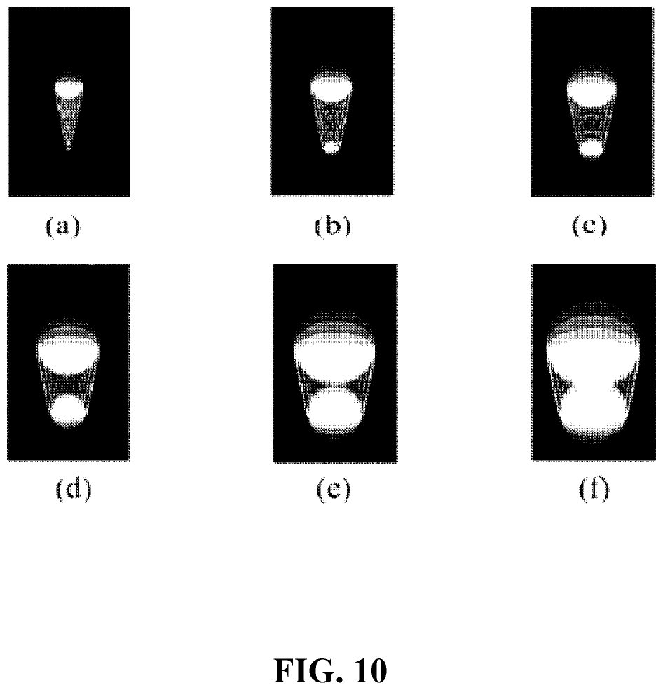

In this example, propagation of shear waves generated by continuous scanning of ARFs in an axial direction is illustrated. An exemplary SWEI system, similar to the SWEI system of EXAMPLE 1, is implemented in which an ultrasound transducer continuously applies ARFs in an axial direction (analogous to axial direction 328). FIG. 10 shows propagation of shear waves generated by continuous scanning of ARFs in an axial direction, consistent with one or more exemplary embodiments of the present disclosure. Subfigures (a) to (f) show propagation of generated shear waves and associated Mach cones at different moments. As FIG. 10 shows, Mach cones are generated in an axial direction and propagate in a lateral direction (analogous to lateral direction 340) through time. Moreover, it can be observed that displacement amplitudes are higher in continuous scanning of ARFs than those in discrete scanning. For example, a maximum displacement of about 31 μm is obtained for continuous scanning, which reduces to about 29 μm during propagation. However, for discrete scanning, the maximum displacement reduces from about 12 μm to about 6 μm.

### EXAMPLE 7

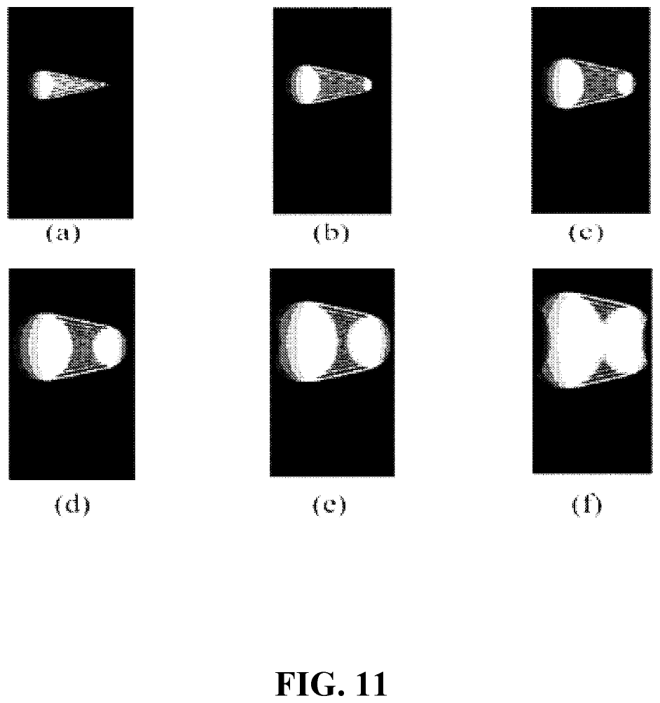

In this example, propagation of shear waves generated by continuous scanning of ARFs in a lateral direction is illustrated. An exemplary SWEI system, similar to the SWEI system of EXAMPLE 1, is implemented in which an ultrasound transducer continuously applies ARFs in a lateral direction (analogous to lateral direction 340). FIG. 11 shows propagation of shear waves generated by continuous scanning of ARFs in a lateral direction, consistent with one or more exemplary embodiments of the present disclosure. Subfigures (a) to (f) show propagation of generated shear waves and associated Mach cones at different moments. As FIG. 11 shows, Mach cones are generated in a lateral direction and propagate in an axial direction (analogous to axial direction 328) through time. Moreover, it can be observed that displacement amplitudes are higher in continuous scanning of ARFs than those of discrete scanning in FIG. 8.

### EXAMPLE 8

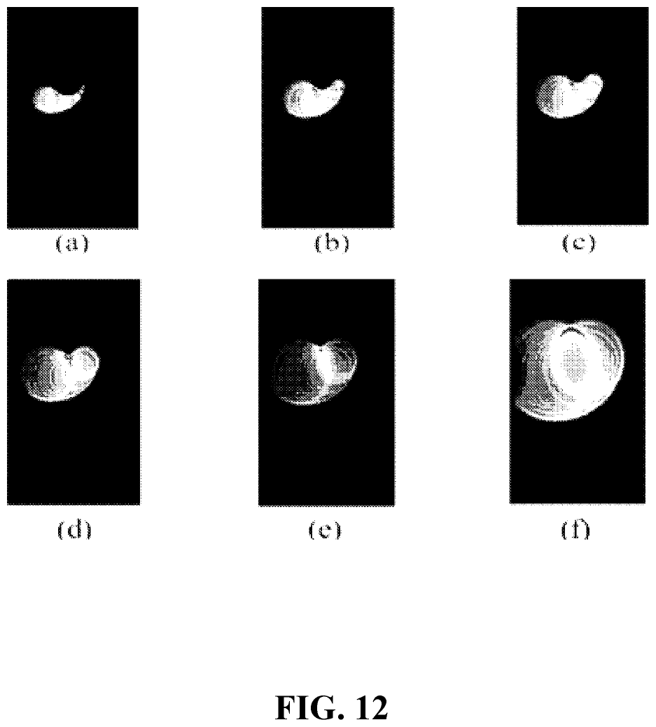

In this example, propagation of shear waves generated by continuous scanning of ARFs through an arch-shaped path is illustrated. An exemplary SWEI system, similar to the SWEI system of EXAMPLE 1, is implemented in which an ultrasound transducer continuously applies ARFs through an arch-shaped path (analogous to arch-shaped path 346). FIG. 12 shows propagation of shear waves generated by continuous scanning of ARFs through an arch-shaped path, consistent with one or more exemplary embodiments of the present disclosure. Subfigures (a) to (f) show propagation of generated shear waves and associated Mach cones at different moments. As FIG. 12 shows, Mach cones are propagate in a special pattern similar to a crescent. Moreover, it can be observed that displacement amplitudes are higher in continuous scanning of ARFs than those of discrete scanning in FIG. 9.

### EXAMPLE 9

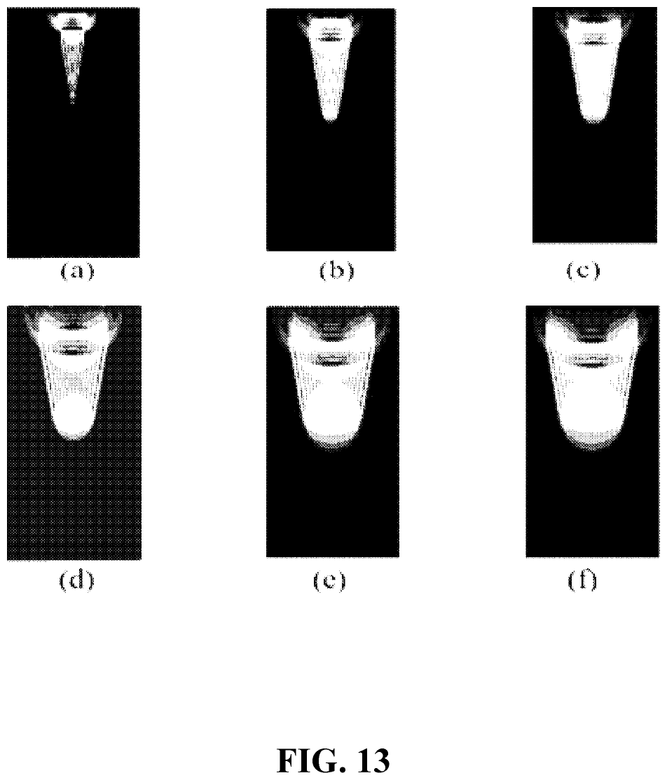

In this example, propagation of shear waves generated by continuous scanning of ARFs in an axial direction along with application of a mechanical force by a vibrator is illustrated. An exemplary SWEI system, similar to the SWEI system of EXAMPLE 1, is implemented in which an ultrasound transducer continuously applies ARFs in an axial direction (analogous to axial direction 328) and simultaneously a mechanical force is applied by a vibrator (analogous to vibrator 204). FIG. 13 shows propagation of shear waves generated by continuous scanning of ARFs in an axial direction and a simultaneous application of a mechanical force, consistent with one or more exemplary embodiments of the present disclosure. Subfigures (a) to (f) show propagation of generated shear waves and associated Mach cones at different moments. As FIG. 13 shows, Mach cones are generated in an axial direction and propagate in a lateral direction (analogous to lateral direction 340) through time. Moreover, it can be observed that displacement amplitudes increase when a mechanical force is also applied for excitation.

### EXAMPLE 10

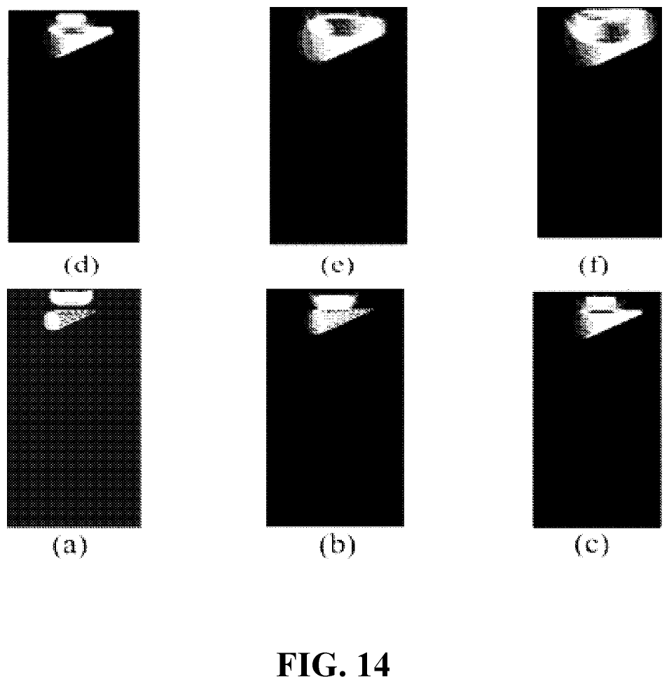

In this example, propagation of shear waves generated by continuous scanning of ARFs in a lateral direction along with application of a mechanical force by a vibrator is illustrated. An exemplary SWEI system, similar to the SWEI system of EXAMPLE 1, is implemented in which an ultrasound transducer continuously applies ARFs in a lateral direction (analogous to lateral direction 340) and simultaneously a mechanical force is applied by a vibrator (analogous to vibrator 204). FIG. 14 shows propagation of shear waves generated by continuous scanning of ARFs in a lateral direction and a simultaneous application of a mechanical force, consistent with one or more exemplary embodiments of the present disclosure. Subfigures (a) to (f) show propagation of generated shear waves and associated Mach cones at different moments. As FIG. 14 shows, Mach cones are generated in a lateral direction and propagate in an axial direction (analogous to axial direction 328) through time. Moreover, it can be observed that displacement amplitudes increase when a mechanical force is also applied for excitation.

### EXAMPLE 11

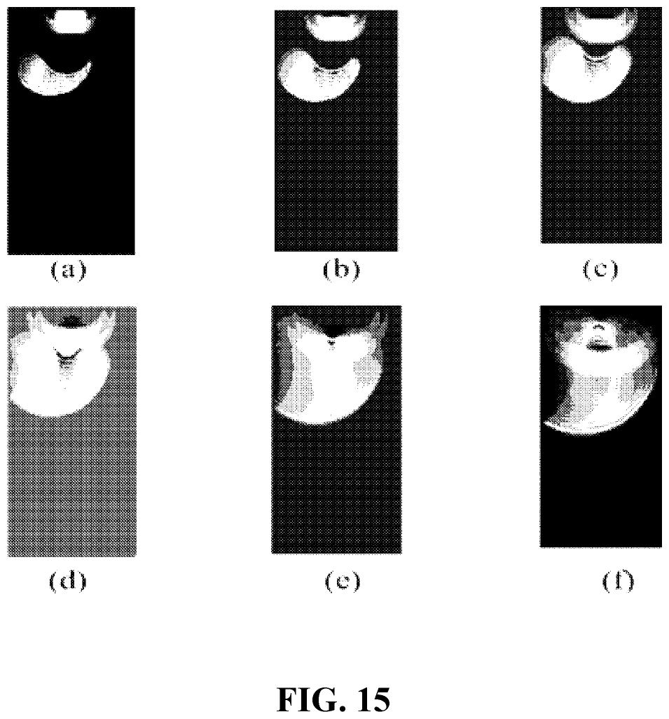

In this example, propagation of shear waves generated by continuous scanning of ARFs through an arch-shaped path along with application of a mechanical force by a vibrator is illustrated. An exemplary SWEI system, similar to the SWEI system of EXAMPLE 1, is implemented in which an ultrasound transducer continuously applies ARFs through an arch-shaped path (analogous to arch-shaped path 346) and simultaneously a mechanical force is applied by a vibrator (analogous to vibrator 204). FIG. 15 shows propagation of shear waves generated by continuous scanning of ARFs through an arch-shaped path and a simultaneous application of a mechanical force, consistent with one or more exemplary embodiments of the present disclosure. Subfigures (a) to (f) show the propagation of generated shear waves and associated Mach cones at different moments. As FIG. 15 shows, Mach cones propagate in a pattern similar to a crescent. Moreover, it can be observed that displacement amplitudes increase when a mechanical force is also applied for excitation.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for generating a source point of shear waves for shear-wave elasticity imaging (SWEI) of an object, the method comprising:
placing an annular array transducer (AAT) on a surface of the object, the AAT comprising a plurality of piezoelectric elements;
vibrating the AAT along a vibration axis utilizing a vibrator when the AAT is placed on the surface of the object;
generating a shear wave in the source point by applying an acoustic radiation force on the source point, applying the acoustic radiation force comprising focusing a first plurality of ultrasound waves propagated from the plurality of piezoelectric elements at a first focus point located at the source point simultaneously with vibrating the AAT;
moving the source point from the first focus point to a second focus point in an axial direction at a supersonic speed by focusing a second plurality of ultrasound waves at the second focus point, the second plurality of ultrasound waves propagated from the plurality of piezoelectric elements;
rotating the AAT in a lateral direction at a supersonic speed utilizing a motor; and
moving the source point along one of an arch-shaped path or a linear path by focusing the AAT at a plurality of focal points simultaneously with rotating the AAT, the plurality of focal points located on the predefined path.

2. A method for generating a source point of shear waves for shear-wave elasticity imaging (SWEI) of an object, the method comprising:
placing an ultrasound transducer on a surface of the object;
vibrating the ultrasound transducer along a vibration axis utilizing a vibrator when the ultrasound transducer is placed on the surface of the object; and
generating a shear wave in the source point by applying an acoustic radiation force on the source point, applying the acoustic radiation force comprising focusing an ultrasonic beam of the ultrasound transducer at a first focus point located at the source point simultaneously with vibrating the ultrasound transducer.

3. The method of claim 2, wherein focusing the ultrasonic beam comprises focusing a first plurality of ultrasound waves at the first focus point, each of the first plurality of ultrasound waves propagated from a piezoelectric element of a plurality of piezoelectric elements of an annular array transducer (AAT).

4. The method of claim 3, further comprising moving the source point from the first focus point to a second focus point in an axial direction by focusing a second plurality of ultrasound waves at the second focus point, the second plurality of ultrasound waves propagated from the plurality of piezoelectric elements.

5. The method of claim 4, wherein moving the source point from the first focus point to the second focus point comprises moving the source point from the first focus point to the second focus point at a supersonic speed.

6. The method of claim 3, further comprising rotating the AAT in a lateral direction at a supersonic speed utilizing a motor.

7. The method of claim 6, further comprising moving the source point along a predefined path by focusing the AAT at a plurality of focal points simultaneously with rotating the AAT, the plurality of focal points located on the predefined path.

8. The method of claim 7, further comprising vibrating the AAT along the vibration axis simultaneously with moving the source point along the predefined path.

9. The method of claim 7, wherein moving the source point along the predefined path comprises moving the source point along one of an arch-shaped path or a linear path.

10. A system for generating a source point of shear waves for shear-wave elasticity imaging (SWEI) of an object, the system comprising:
an ultrasound transducer configured to be placed on a surface of the object;
a vibrator coupled with the ultrasound transducer;
a memory having processor-readable instructions stored therein; and
one or more processors configured to access the memory and execute the processor-readable instructions, which, when executed by the one or more processors configures the processor to perform a method, the method comprising:
vibrating the ultrasound transducer along a vibration axis utilizing the vibrator when the ultrasound transducer is placed on the surface of the object; and
generating a shear wave in the source point by applying an acoustic radiation force on the source point, applying the acoustic radiation force comprising focusing an ultrasonic beam of the ultrasound transducer at a first focus point located at the source point simultaneously with vibrating the ultrasound transducer.

11. The system of claim 10, wherein the ultrasound transducer comprises an annular array transducer (AAT) comprising a plurality of piezoelectric elements.

12. The system of claim 11, wherein focusing the ultrasonic beam comprises focusing a first plurality of ultrasound waves at the first focus point, each of the first plurality of ultrasound waves propagated from a piezoelectric element of the plurality of piezoelectric elements.

13. The system of claim 12, wherein the method further comprises moving the source point from the first focus point to a second focus point in an axial direction by focusing a second plurality of ultrasound waves at the second focus point, the second plurality of ultrasound waves propagated from the plurality of piezoelectric elements.

14. The system of claim 13, wherein moving the source point from the first focus point to the second focus point comprises moving the source point from the first focus point to the second focus point at a supersonic speed.

15. The system of claim 12, further comprising a motor coupled with the AAT.

16. The system of claim 15, wherein the method further comprises rotating the AAT in a lateral direction at a supersonic speed utilizing the motor.

17. The system of claim 16, wherein the method further comprises moving the source point along a predefined path by focusing the AAT at a plurality of focal points simultaneously with rotating the AAT, the plurality of focal points located on the predefined path.

18. The system of claim 17, wherein the method further comprises vibrating the AAT along the vibration axis simultaneously with moving the source point along the predefined path.

19. The system of claim 17, wherein moving the source point along the predefined path comprises moving the source point along one of an arch-shaped path or a linear path.
